(19) United States
(12) Patent Application Publication
(10) Pub. No.: US 2008/0103427 A1
(43) Pub. Date: May 1, 2008

(54) BLOOD PURIFICATION DEVICE

(75) Inventors: Masahiro Toyoda, Shizuoka (JP); Tomoya Murakami, Shizuoka (JP)

Correspondence Address: DARBY & DARBY P.C., P.O. BOX 770, Church Street Station, New York, NY, 10008-0770, US

(73) Assignee: NIKKISO CO., LTD., Tokyo (JP)

(21) Appl. No.: 11/863,493

(22) Filed: Sep. 28, 2007

(30) Foreign Application Priority Data
Nov. 1, 2006 (JP) 2006-297882

(51) Int. Cl. A61M 1/14

(52) U.S. Cl. 604/5.04; 604/5.01

(57) ABSTRACT

A blood purification device capable of performing an ideal blood purification treatment which accounts for blood recirculation is provided. That is, a blood purification device is furnished with a blood circuit containing an arterial blood circuit and a venous blood circuit for circulating blood outside the body, a dialyzer for purifying blood flowing in the blood circuit, hematocrit sensors for measuring the hematocrit value of blood circulating outside the body in the blood circuit, and a recirculation rate derivation means for obtaining a recirculation rate, and further provided with a true value derivation means for obtaining a patient's true hematocrit value based on the recirculation rate obtained by the recirculation rate derivation means.

### CROSS-REFERENCE TO PRIOR RELATED APPLICATION

This application claims priority under 35 U.S.C. 119 to Japanese Patent Application No. 2006-297882, filed on Nov. 1, 2006. The content of the Japanese application is incorporated herein by reference in its entirety.

### FIELD OF THE INVENTION

The present invention relates to a blood purification device for circulating and purifying patient blood outside the body.

### BACKGROUND OF THE INVENTION

In blood purification treatments such as dialysis, a blood circuit containing a flexible tube is generally used to circulate blood outside a patient's body. The blood circuit is composed primarily of an arterial blood circuit attached to the end of an arterial puncture needle for collecting blood from a patient, and a venous blood circuit attached to the end of a venous puncture needle for returning blood to the patient. A dialyzer is interposed between the arterial blood circuit and the venous blood circuit to purify blood circulating outside the body.

A plurality of hollow fibers are disposed inside the dialyzer, constituted such that blood passes through the interior of the hollow fibers, while dialysate can be caused to flow on the outside thereof (between the outer circumferential surface of the hollow fibers and the inner circumferential surface of the casing). Very small holes (pores) are formed on the wall surface of the hollow fibers to constitute a blood purification membrane. Waste matter and the like in the blood passing through the interior of the hollow fibers is transmitted through the blood purification membrane and expelled into the dialysate, and blood which has been purified by the removal of waste is returned to the patient's body. An ultrafiltration pump for removing water from the patient's blood is disposed within the dialysis device and water is removed during dialysis treatment.

When an arterial puncture needle and a venous puncture needle puncture a patient's shunt (a site at which an artery is surgically connected to a vein) and the surrounding area and blood is circulated outside the body, blood recirculation can occur whereby blood from the subject venous puncture needle which has been purified and returned to the patient's body is once again guided back from the arterial puncture needle without passing through the patient's organs. When this type of recirculation occurs, the purified blood is yet further circulated outside the body, reducing by that amount the volume of blood requiring purification which can flow outside the body and leading to deleterious degrading of blood purification efficiency.

Dialysis devices have therefore been earlier proposed whereby a particular peak is imparted to a change in the concentration of blood circulated outside the body by driving an ultrafiltration pump suddenly and for a short duration of time. Blood recirculation is detected using this event as a marker (see, for example, Japanese Laid Open Patent Application Publication No. 2000-502940). According to the dialysis device disclosed in this citation, a blood concentration detection sensor (hemoglobin concentration detection sensor) is disposed on an arterial blood circuit, and blood recirculation in dialysis treatment is sensed by detecting a particular peak using that sensor.

Furthermore, previous proposals have been offered whereby in addition to a sensor disposed in the arterial blood circuit, a sensor is also similarly provided on a venous blood circuit, thereby permitting verification of whether a particular peak was imparted to a change in blood concentration, and also reducing the number of parameters for obtaining the proportion of recirculated blood so that blood recirculation detection can be performed reliably and accurately (see, for example, Japanese Unexamined Patent Application Publication No. 2006-087907).

### SUMMARY OF THE INVENTION

However, since the above-described prior blood purification devices merely detect blood recirculation, the problem has remained that it was still difficult to perform blood purification treatment which takes said blood recirculation into account. Even if blood recirculation is detected, in other words, it is difficult for a doctor or other health practitioner to predict in real time what kind of influence it will have on the circulating blood volume rate of change (ΔBV), for example, which is an indicator of patient condition, or on the clearance value, which is an indicator of the volume and efficiency of dialysis performed by the dialyzer, such that ideal blood purification treatment which accounts for the subject blood recirculation cannot be performed.

The present invention was undertaken in light of these factors, and provides a blood purification device capable of performing an ideal blood purification treatment which accounts for blood recirculation.

One aspect of the invention is a blood circuit containing an arterial blood circuit and a venous blood circuit for circulating collected patient blood outside the body, a blood pump disposed on the arterial blood circuit, a blood purification means connected between the arterial blood circuit and the venous blood circuit for purifying blood flowing in said blood circuit, a concentration measurement means for measuring a blood indicator indicating the concentration of blood circulating in the blood circuit outside the body, and a recirculation rate derivation means capable of obtaining a recirculation rate showing the proportion of recirculated blood, which is blood returned to a patient from the venous blood circuit and once again directed to the arterial blood circuit, vs. blood flowing in the arterial blood circuit, and is furnished with a true value derivation means capable of obtaining a patient's true blood indicator based on a recirculation rate obtained by the recirculation rate derivation means.

In another aspect of the invention, the concentration measurement means in the blood purification device described above is disposed on the blood circuit and contains a hematocrit sensor for measuring the hematocrit value of blood flowing through said blood circuit, and the true blood indicator to be obtained from the true value derivation means is a hematocrit value.

In another aspect of the invention, the circulating blood volume rate of change, which is an indicator of patient condition, is calculated based on the true hematocrit value obtained by the true value derivation means in the blood purification device described immediately above.

In another aspect of the invention, the concentration measurement means in the blood purification device described above are respectively disposed on the arterial blood circuit and the venous blood circuit in the blood circuit.

In another aspect of the invention, the blood purification means described above contains a dialyzer for introducing or expelling dialysate via a dialysis membrane, and the concentration measurement means can measure a blood indicator showing blood concentration from dialysate pressure, which is the pressure of dialysate expelled from said dialyzer.

In another aspect of the invention, the concentration measurement means in the blood purification device described above is disposed on the blood circuit, and contains a hematocrit sensor for measuring the hematocrit value of blood flowing in said blood circuit and a solute concentration measurement sensor for measuring the solute concentration of blood flowing in said blood circuit. The true blood indicator to be obtained by the true value derivation means is the solute concentration.

In another aspect of the invention, the blood purification means of the blood purification device described above contains a dialyzer which either introduces or expels dialysate via a dialysis membrane. A clearance value indicating the dialysis volume and efficiency of said dialyzer is calculated based on the true solute concentration obtained by the true value derivation means.

According to one feature of the invention, a patient's true blood indicator can be obtained by a true value derivation means based on a recirculation rate obtained by a recirculation derivation means, thereby enabling the performance of an ideal blood purification treatment which accounts for blood recirculation.

According to another feature of the invention, the true blood indicator obtained by the true value derivation means is a hematocrit value, and therefore the hematocrit value and each of the indicators obtained from the hematocrit value can be accurately obtained.

According to another feature of the invention, the circulating blood volume rate of change, which is an indicator of patient condition, is calculated based on the true hematocrit value obtained by the true value derivation means, and therefore the circulating blood volume rate of change can be obtained with good accuracy in real time, and by using this rate as an indicator during blood purification treatment, an ideal blood purification treatment which accounts for blood recirculation can thus be performed.

According to another feature of the invention, the concentration measurement means are respectively disposed in the arterial blood circuit and the venous blood circuit within the blood circuit. Therefore, compared to a system in which a concentration measurement means is disposed on only one of the arterial blood circuit or the venous blood circuit, the number of parameters for seeking the proportion of recirculated blood (blood recirculation rate) can be reduced, and therefore the recirculation rate can be reliably and accurately obtained, and a true blood indicator can be more rapidly obtained.

According to another feature of the invention, the concentration measurement means enables a blood indicator showing blood concentration to be measured from dialysate pressure, which is the pressure of the dialysate expelled from the dialyzer, and therefore it is not necessary to provide a concentration measurement means on the blood circuit side.

According to another feature of the invention, the true blood indicator obtained by the true value derivation means is the solute concentration, and therefore the solute concentration and each type of indicator obtained from this solute concentration can be accurately obtained.

According to another feature of the invention, the clearance value, which is an indicator of dialysis volume and efficiency by the dialyzer, is calculated based on the true solute concentration obtained by the true value derivation means, and therefore the clearance value can be accurately obtained in real time, and using this value as an indicator during blood purification treatment enables the performance of ideal blood purification treatment which accounts for blood recirculation.

### BRIEF DESCRIPTION OF THE DRAWINGS

FIG. 1 is an overall schematic showing a blood purification device related to an embodiment of the present invention.

FIG. 2 is a schematic showing the dialysis device main unit in a blood purification device.

FIG. 3 is a graph showing control of the ultrafiltration pump in a blood purification device. The graph shows that water removal is performed suddenly and in a short time duration.

FIG. 4 is a graph showing the change in the hematocrit value detected by the second hematocrit sensor in a blood purification device.

FIG. 5 is a graph showing the change in the hematocrit value (when recirculation is occurring) detected by the first hematocrit sensor in a blood purification device.

FIG. 6 is a block diagram showing the interconnections among a first hematocrit sensor, a second hematocrit sensor, a computing means, a true value derivation means, a derivation means for the circulating blood volume rate of change, and a display means.

FIG. 7 is an explanatory diagram schematically showing the case in which recirculated blood is present in a blood purification device.

FIG. 8 is an explanatory diagram schematically showing the case in which recirculation occurs in a blood purification device related to another embodiment of the present invention.

FIG. 9 is a block diagram showing the interconnections among a first solute concentration measurement sensor, a second solute concentration measurement sensor, a computation means, a true value derivation means, and a clearance value derivation means.

### DETAILED DESCRIPTION OF THE INVENTION

Below we explain embodiments of the present invention in concrete terms with reference to drawings.

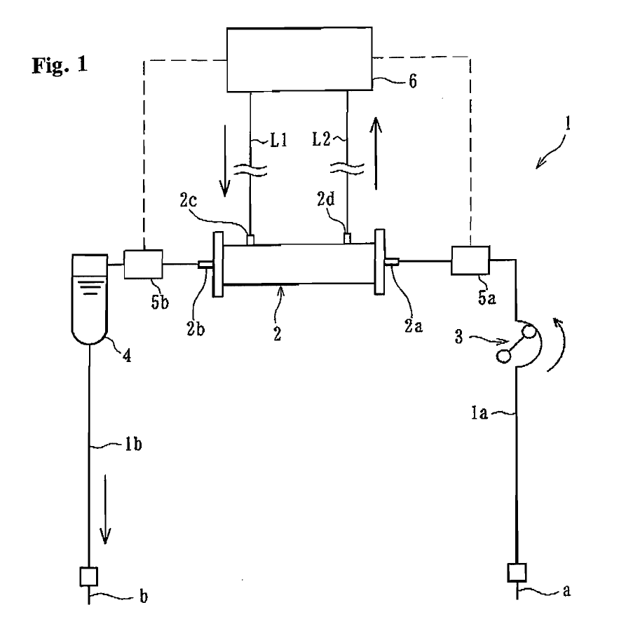

The blood purification device of an embodiment purifies patient blood while circulating it outside the body, and is therefore applied to a dialysis device used in dialysis treatment. The dialysis device, as shown in FIG. 1, is composed primarily of a blood circuit 1 to which a dialyzer 2 is connected, and a dialysis device main unit 6 for supplying dialysate to the dialyzer 2 and removing water. The blood circuit 1, as shown in the same figure, is composed primarily of an arterial blood circuit 1a and a venous blood circuit 1b made up of flexible tubing. The dialyzer 2 is connected between the arterial blood circuit 1a and the venous blood circuit 1b.

An arterial puncture needle a is connected to the arterial blood circuit 1a, and a perfusion blood pump 3 and a first hematocrit sensor (concentration measurement means) 5a are disposed midway thereupon. At the same time, a venous puncture needle b is connected at the end thereof to a venous blood circuit 1, and a second hematocrit sensor 5b (concentration measurement means) and a drip chamber 4 for removing air bubbles are connected midway thereto.

In other words, the first hematocrit sensor 5a and the second hematocrit sensor 5b which constitute the concentration measurement means in the present embodiment are respectively disposed on the arterial blood circuit 1a and the venous blood circuit 1b, such that the blood indicator (specifically the hematocrit value) which indicates the concentration of blood circulating in the blood circuit 1 outside the body can be measured in real time. Here the hematocrit value is an indicator of blood concentration; specifically it is expressed as a bulk ratio of red blood cells to total blood.

When the blood pump 3 is driven in a state in which the arterial puncture needle a and the venous puncture needle b are puncturing a patient, the patient's blood reaches the dialyzer 2 through the arterial blood circuit 1a, and blood purification is accomplished by the dialyzer 2. Air bubbles are then removed in the drip chamber 4 and the blood returns to the patient's body via the venous blood circuit 1b. In other words, the patient's blood is caused to circulate outside the body in the blood circuit 1 and is purified by the dialyzer 2.

A blood introduction port 2a, a blood expelling port 2b, a dialysate introduction port 2c, and a dialysate expelling port 2d are formed on the casing portion of the dialyzer 2. Of these, the base end of the arterial blood circuit 1a is connected to the blood introduction port 2a, and the base end of the venous blood circuit 1b is connected to the blood expelling port 2b. The dialysate introduction port 2c and the dialysate expelling port 2d are respectively connected to a dialysate introduction line L1 and a dialysate expelling line L2.

A plurality of hollow fibers are housed within the dialyzer 2. The interiors of these hollow fibers serve as a blood flow path, while the space between the outer circumferential surface of the hollow fibers and the inner circumferential surface of the casing portion serves as a dialysate flow path. A number of very small holes (pores) are formed so as to penetrate the outer circumferential surface and inner circumferential surface of the hollow fibers and form a hollow fiber membrane. Impurities and the like in the blood can pass into the dialysate through this membrane.

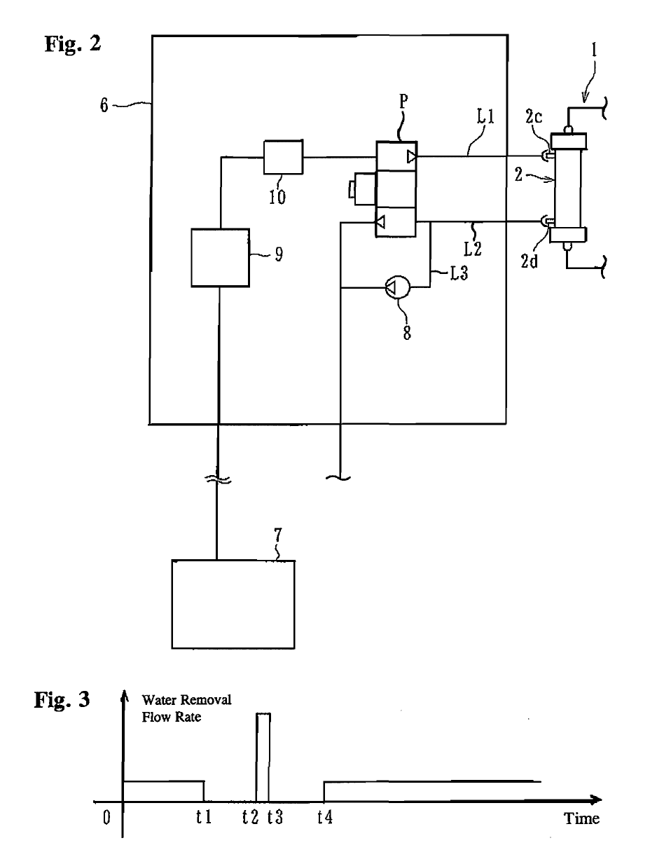

As shown in FIG. 2, the dialysis device main unit 6 is composed primarily of a duplex pump P formed across the dialysate introduction line L1 and the dialysate expelling line L2, a bypass line L3 connected to the dialysate expelling line L2 and circumventing the duplex pump P, and an ultrafiltration pump 8 connected to this bypass line L3. One end of the dialysate introduction line L1 is connected to the dialyzer 2 (dialysate introduction port 2c), and the other end thereof is connected to a dialysate supply device 7 for preparing dialysate of a predetermined concentration.

One end of the dialysate expelling line L2 is connected to the dialyzer 2 (dialysate expelling port 2d), and the other end thereof is connected to a fluid disposal means which is not shown. After dialysate supplied from the dialysate supply device 7 reaches the dialyzer 2 through the dialysate introduction line L1, it is sent to the fluid disposal means through the dialysate expelling line L2 and the bypass line L3. Note that reference numerals 9 and 10 in this figure indicate a humidifier and a degassing means connected to the dialysate introduction line L1.

The purpose of the ultrafiltration pump 8 is to remove water from patient blood flowing through the dialyzer 2. In other words, because the duplex pump P is of the fixed-volume type, driving the ultrafiltration pump 8 results in a greater capacity for fluid removal by the dialysate expelling line L2 than the volume of dialysate introduced by the dialysate introduction line L1. Therefore, water is removed only from that excess capacity blood. Note that water can also be removed from patient blood by a means other than the ultrafiltration pump 8 (e.g. by a device which makes use of a so-called balancing chamber or the like).

In addition to performing the water removal required for dialysis treatment, the ultrafiltration pump 8 of the present embodiment can also remove water suddenly and in a short time duration. That is, when water removal being conducted at a fixed speed during dialysis treatment is temporarily stopped (with circulation outside the body taking place) and the measured hematocrit value has stabilized, driving the ultrafiltration pump 8 suddenly and for a short duration to remove water can impart a peak particular to the resulting change in blood concentration (hematocrit value) for that period. Here the words “suddenly and for a short duration” in the present invention refer to a degree of and time within which a pulse applied after blood passes through the circuit can be verified; “particular to” means that it can be distinguished from fluctuation patterns due to other factors caused by pump fluctuations, patient body movements, and the like.

More concretely, water removal at a fixed speed (normal water removal) is stopped at a time t1, as shown in FIG. 3, and when the hematocrit value being measured subsequently reaches a stable time t2, the ultrafiltration pump 8 is driven at a speed higher than normal until a time t3. The period from times t2 to t3 is assumed to be extremely short. Water can thus be removed in a sudden and short time period compared to normal water removal, and a particular peak can be assigned to the hematocrit value, as shown for example in FIG. 4.

As previously discussed, the first hematocrit sensor 5a and the second hematocrit sensor 5b are respectively disposed on the arterial blood circuit 1a and the venous blood circuit 1b. They detect the concentration of blood (specifically the hematocrit value) flowing through these circuits. A hematocrit value indicating patient blood concentration can be detected by providing a light emitting device such as an LED and a light receiving device such as a photo diode, and illuminating blood with light from the light-emitting device as well as receiving light passing through the blood or reflected therefrom in the light-receiving device.

Specifically, a hematocrit value showing blood concentration is obtained based on an electrical signal output from the light receiving device. That is, each component of blood such as red blood cells and plasma has particular light absorption characteristics, and the relevant hematocrit values can be obtained by using these characteristics to electro-optically quantify the red blood cells needed to measure a hematocrit value. More specifically, near-infrared light irradiated from a light emitting diode is made incident on the blood where it is affected by absorption and scattering before being received by the light-receiving device. The light's absorption scattering rate is analyzed from the intensity of that received light, and a hematocrit value is calculated.

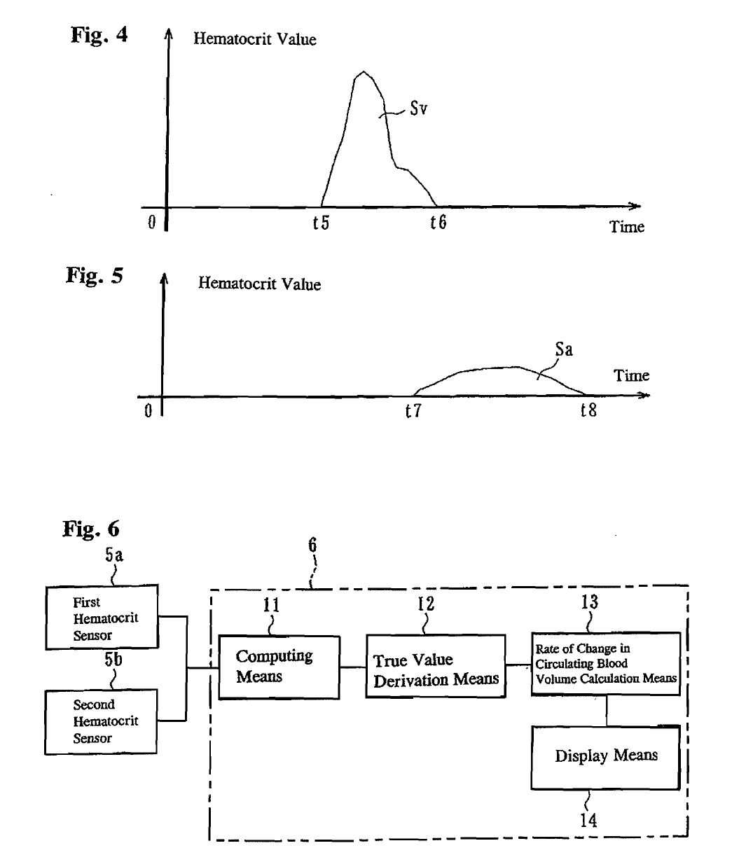

The first hematocrit sensor 5a constituted as described above is disposed on the arterial blood circuit 1a, and therefore the hematocrit value of blood collected from patients via the arterial puncture needle during dialysis treatment is detected, while at the same time the hematocrit sensor 5b is disposed on the venous blood circuit 1b. That blood is thus purified by the dialyzer 2, and a hematocrit value for the blood returned to the patient is detected. In other words, the particular peak imparted by the ultrafiltration pump 8 is first detected in the second hematocrit sensor 5b (see FIG. 4), and if there is 30 subsequently blood which again reaches the arterial blood circuit 1a such that recirculation occurs, the first hematocrit sensor 5a can detect the particular peak remaining in that recirculation blood (see FIG. 5).

Therefore, in addition to the second hematocrit sensor 5b's ability to verify that the ultrafiltration pump 8 has imparted a particular peak, the presence of recirculating blood can also be detected by the first hematocrit sensor 5a. In other words, verification can be performed as to whether a particular peak has been imparted by the ultrafiltration pump 8, and therefore compared to the case in which the hematocrit sensor is disposed on the arterial blood circuit 1a only, a more reliable and accurate blood recirculation detection can be performed.

Moreover, the above first hematocrit sensor 5a and second hematocrit sensor 5b are, as shown in FIG. 6, electrically connected to a computing means 11 disposed on the dialysis device main unit 6. The computing means 11 is electrically connected to a display means 14 such as a liquid crystal display screen via a true value derivation means 12 and a circulating blood volume rate of change calculation means 13. The computing means 11 contains, for example, a microprocessor or the like. The hematocrit values (particular peaks) detected by the first hematocrit sensor 5a and the second hematocrit sensor 5b are compared, and the fraction of recirculated blood in the blood flowing in the arterial blood circuit 1a (i.e., the fraction of recirculated blood, which is blood returned from the venous blood circuit 1b to the patient which is again guided to the arterial blood circuit 1a, vs. the total blood flowing in the arterial blood circuit 1a; referred to below as the recirculation rate) can be computed.

Specifically, when blood recirculation occurs, predictions are made of the time from the assignment of a particular peak by the ultrafiltration pump 8 up until the blood reaches the second hematocrit sensor 5b (t5 in FIG. 4) and the time until that blood reaches the first hematocrit sensor 5a by recirculation (t7 in FIG. 5), a particular peak is imparted by the ultrafiltration pump 8, and the computing means 11 compares the hematocrit value detected by the second hematocrit sensor 5b when time t5 has elapsed and the hematocrit value detected by the first hematocrit sensor 5a when time t7 has elapsed.

Predicting the time t5 at which the blood reaches the second hematocrit sensor 5b and the time t7 at which that blood recirculates to reach the first hematocrit sensor 5a thus enables cardio-pulmonary recirculation (the phenomenon by which purified blood is drawn out of the body after passing through only the heart or the lungs, without passing through tissue, organs, etc.) to be distinguished from the recirculation which is the object of measurement. Note that as an alternative to the above method, the computing means 11 can also be made to recognize that hematocrit values detected by the first hematocrit sensor 5a and the second hematocrit sensor 5b have exceeded a predetermined numerical value, and a comparison made between hematocrit values surpassing said numerical values.

Changes in the hematocrit values of the first hematocrit sensor 5a and the second hematocrit sensor 5b are obtained based on the graph of time-hematocrit values shown in FIGS. 4 and 5, and the areas of the time portions (change portions) to be compared as described above are calculated by a mathematical method such as integration. For example, if Sv is the portion changed according to the second hematocrit sensor 5b (the portion from t5 to t6 in FIG. 4), and Sa is the portion changed according to the first hematocrit sensor 5a (the portion from t7 to t8 in FIG. 5), a recirculation rate AR is obtained by the following equation:

AR(%)=Sa/SV×100

Here, taking into account the fact that blood assigned a particular peak diffuses in the process of flowing from the second hematocrit sensor 5b to the first hematocrit sensor 5a, the time for the portion changed according to the first hematocrit sensor 5a (the time gap from t7 to t8) is set to be larger than the time for the portion changed according to the second hematocrit sensor 5b (the time interval from t5 to t6).

The ultrafiltration pump 8 which imparts the particular peak and the computing means 11 constitute the recirculation rate derivation means in the present invention by which the recirculation rate is thus obtained. The recirculation rate obtained by the computing means 11 is sent to the true value derivation means 12 containing a microprocessor or the like to obtain the true patient hematocrit value (blood indicator).

That is, because the first hematocrit sensor 5a measures the hematocrit value of blood which has not been purified by the dialyzer 2, the measured value taken by the first hematocrit sensor 5a must be used as the patient hematocrit value if no blood recirculation is present, but if blood recirculation is occurring, the influence thereof may mean that the value measured by the first hematocrit sensor 5a is not necessarily the patient's true hematocrit value, and the true hematocrit value can be obtained by the true value derivation means 12 in order to account for this effect.

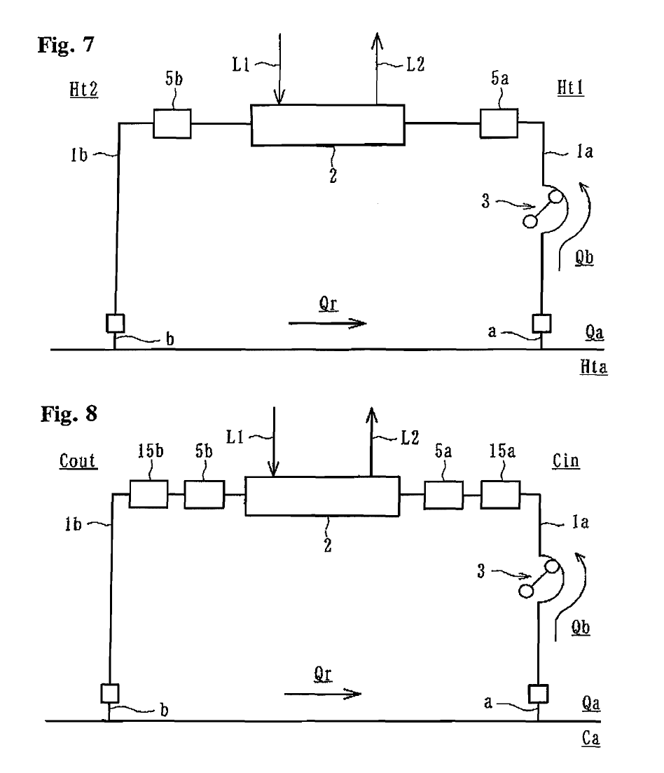

Specifically, assuming as shown in FIG. 7, a shunt (e.g., the blood vessel short circuit portion on the body side) blood flow volume (shunt flow volume) Qa, a blood flow volume flowing in blood circuit 1 arising from the action of the blood pump 3 (blood pump flow volume) Qb, and a recirculation blood flow volume (recirculation flow volume) Qr, and setting a hematocrit value Ht1 measured by the first hematocrit sensor 5a, a hematocrit value Ht2 measured by the second hematocrit sensor 5b, and a pre-purification shunt hematocrit value (true hematocrit value) Hta, the following relationship can be expressed:

Ht1×Qb=Hta×Qa+Ht2×Qr (Eq. 1)

(where Qa≦Qb; Qb=Qa+Qr)

Given here that Qb=Qa+Qr, we can apply the expression Qa=Qb−Qr, and using a recirculation rate AR, the recirculation rate (AR)=Qr/Qb, which permits the expression Qr=AR×Qb. Substituting these in the expression above, we have:

Ht1×Qb=Hta×Qb×(1−AR)+Ht2×Qb×AR (Eq. 2)

From Equation 2 above, we obtain an expression for the true hematocrit value Hta:

Hta=(Ht1−Ht2×AR)/(1−AR) (Eq. 3)

That is, the true hematocrit value Hta can be obtained from the above Equation 3, given that the first hematocrit sensor 5a measured value Ht1, the second hematocrit sensor 5b measured value Ht2, and the AR obtained by computing means 11 are known parameters.

According to the present embodiment, a true patient blood indicator (hematocrit value) can be obtained by the true value derivation means 12 based on the recirculation rate obtained by the computing means 11 (the recirculation rate derivation means), thereby enabling an ideal blood purification treatment which accounts for blood recirculation. Since the true blood indicator to be obtained by the true value derivation means 12 is the hematocrit value, that hematocrit value and the various indicators obtained from that hematocrit value can be accurately obtained.

Furthermore, the true hematocrit value obtained above is sent to a circulating blood volume rate of change calculation means 13 containing a microprocessor or the like so as to enable calculation of the circulating blood volume rate of change (ΔBV), which is an indicator of patient condition. This circulating blood volume rate of change (ΔBV) can be obtained by the following operation:

(hematocrit value at the beginning of dialysis (Ht(0))−hematocrit value at the time of measurement (Ht(t)))/hematocrit value at the time of measurement (Ht(t))×100.

Therefore, the circulating blood volume rate of change (ΔBV) can be calculated by substituting the true hematocrit value (Ht (0) and Ht (t)) in this expression.

Therefore, according to the present embodiment the circulating blood volume rate of change (ΔBV) is calculated by the circulating blood volume rate of change calculation means 13 based on the true hematocrit value obtained by the true value derivation means 12, thus permitting an accurate circulating blood volume rate of change (ΔBV) to be obtained in real time. By using this value as an indicator during blood purification treatment an ideal blood purification treatment which accounts for blood recirculation can be performed. The circulating blood volume rate of change (ΔBV) calculated by the circulating blood volume rate of change calculation means 13 is displayed in real time on a display means 14.

According to the present embodiment, the first hematocrit sensor 5a and the second hematocrit sensor 5b functioning as concentration measurement means are respectively disposed on the arterial blood circuit 1a and the venous blood circuit 1b in the blood circuit 1, and therefore the number of parameters for obtaining the recirculation rate is reduced compared to the case in which the hematocrit sensors are disposed as concentration measurement means on only one of either the arterial blood circuit 1a or the venous blood circuit 1b, the recirculation rate can be obtained more reliably and accurately, and the true blood indicator can be obtained more quickly.

Therefore, in the present embodiment the first hematocrit sensor 5a and the second hematocrit sensor 5b functioning as concentration measurement means are respectively disposed as noted above on the arterial blood circuit 1a and the venous blood circuit 1b, but can instead be disposed on the venous blood circuit 1b only. In such cases, using a volume of water removed by the ultrafiltration pump 8 Quf (a known parameter), the true hematocrit value Hta can be obtained from the following expression:

Hta={Ht1−(Ht1×AR×Qb)/(Qb−Quf)}/(1−AR)

Moreover, in the present embodiment the first hematocrit sensor 5a and the second hematocrit sensor 5b functioning as concentration measurement means are disposed on the blood circuit 1 side, but alternatively the concentration measurement means can also measure a blood indicator (hematocrit value, hemoglobin concentration, or the like) showing blood concentration from dialysate pressure, which is the pressure of the dialysate derived from the dialyzer 2.

Specifically, the pressure differential between the blood flow path in the dialyzer 2 and the dialysate flow path (the cross-membrane pressure differential in the dialyzer 2 hollow fibers membrane (dialysis membrane)) is grasped from the difference between the venous pressure and the dialysate pressure, while the fact that this cross-membrane pressure differential changes due to the concentration of patient blood circulating outside the body enables this cross-membrane pressure differential to be used as a blood indicator for indicating the concentration of blood circulating outside the body. In such cases, there is no need to provide a concentration measurement means on the blood circuit 1 side.

Next we explain another embodiment of the present invention.

The present blood purification device purifies a patient's blood while circulating it outside the body. It is composed primarily of a blood circuit 1 connected to a dialyzer 2 as a blood purification means and a dialysis device main unit 6 for removing water while supplying dialysate to the dialyzer 2, and is applied to a dialysis device used for dialysis treatment. Note that those constituent elements which are the same as those in the previous embodiment are assigned the same reference numerals, and a detailed explanation thereof is omitted.

In addition to hematocrit sensors 5a and 5b for measuring the hematocrit value of blood flowing in these blood circuits, a first solute concentration measuring sensor 15a and a second solute concentration measuring sensor 15b are respectively disposed on the arterial blood circuit 1a and the venous blood circuit 1b of the present embodiment as shown in FIG. 8. The first solute concentration measuring sensor 15a and the second solute concentration measuring sensor 15b respectively measure the blood solute concentration (urea concentration, etc.) of the arterial blood circuit 1a and the venous blood circuit 1b.

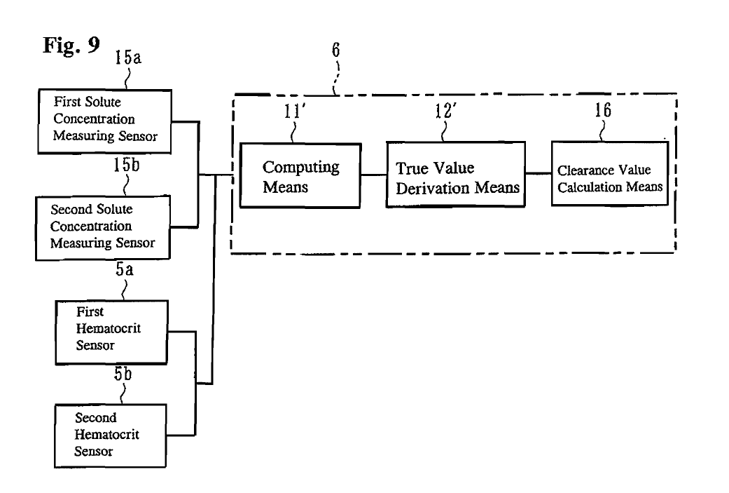

Furthermore, the first solute concentration measuring sensor 15a and the second solute concentration measuring sensor 15b are, as shown in FIG. 9, electrically connected 5 to a computing means 11′, and this computing means 11′ is electrically connected via a true value derivation means 12′ to a clearance value calculating means 16. The computing means 11′, as in the previous embodiment, contains an ultrafiltration pump 8 capable of imparting a particular peak, and a recirculation rate derivation means. The recirculation rate (AR) is obtained by these means. Note that the recirculation rate (AR) derivation method is the same as in the previous embodiment.

The recirculation rate obtained by the computing means 11′ is sent to a true value derivation means 12′ containing a microprocessor or the like to obtain the true patient solute concentration (blood indicator). Specifically, assuming as shown in FIG. 8 a flow volume of blood (shunt flow volume) in the shunt (e.g., the body side blood vessel short circuit portion) Qa and a blood flow volume flowing in the blood circuit 1 arising from the action of the blood pump 3 (blood pump flow volume) Qb, and a recirculation blood flow volume (recirculation flow volume) Qr, and setting the solute concentration measured by the first solute concentration measuring sensor 15a as Cin and the solute concentration measured by the second solute concentration measuring sensor 15b as Cout, with a pre-purification blood solute concentration (true solute concentration) Ca, this can be expressed by the relationship below, due to the material balance expression:

Cin×Qb=Ca×Qa+Cout×Qr (Eq. 4)

(where Qa≦Qb; Qb=Qa+Qr)

Here Qb=Qa+Qr, so we can state that Qa=Qb−Qr. Therefore, if the recirculation rate is AR, the recirculation rate (AR)=Qr/Qb, and we can state that Qr AR×Qb. Substituting these in Equation 4 above, we have:

Cin=Ca×(1−AR)+Cout×AR (Eq. 5)

From the above Equation 5, the expression for obtaining the true solute concentration Ca is as follows:

Ca=(Cin−Cout×AR)/(1−AR) (Eq. 6)

In other words, the first solute concentration measuring sensor 15a measurement value Cin, the second solute concentration measuring sensor 15b measurement value Cout, and the AR obtained by the computing means 11′ are known parameters, and therefore the true solute concentration Ca can be obtained by the above Equation 6.

According to the present embodiment, a true patient blood indicator (solute concentration) can be obtained by the true value derivation means 12′ based on the recirculation rate obtained by the computing means 11′ (recirculation rate derivation means) as in the previous embodiment, and therefore an ideal blood purification treatment which accounts for the subject blood recirculation can be performed. The true blood indicator to be obtained by the true value derivation means 12′ is a solute concentration, and therefore that solute concentration and the various indicators obtained from that solute concentration can be accurately obtained.

Moreover, the true solute concentration obtained as described above is sent to a clearance value calculation means containing a microcomputer or the like to calculate a clearance value K, which is an indicator showing the dialysis volume and efficiency of the dialyzer 2. This clearance value K is a parameter which primarily indicates the material removal performance of the dialyzer 2 and how many mL of blood have passed through the dialyzer 2.

This clearance value depends on the membrane surface area, the blood flow volume (blood flow volume circulating outside the body), membrane properties, etc., and is therefore a parameter particular to that dialyzer (a particular value) which should be known beforehand. Generalizing the clearance value K in a mathematical expression for the case where there is no water removal, we have:

K=(Cin−Cout)/Cin×Qb (Eq. 7)

In the above Equation 7, Cin (solute concentration measured by the first solute concentration measuring sensor 15a) is equal to Ca (true solute concentration) when there is no recirculation, but when there is recirculated blood, Cin and Ca are not equal, thereby causing an error in the clearance value as determined by the above general expression. Therefore, in the present embodiment a clearance value K0 (true clearance value), which is an indicator showing the dialyzer 2 dialysis volume and efficiency, is calculated based on the true solute concentration obtained by the true value derivation means 12′.

When recirculated blood is present, the volume of solute (urea) removed is obtained by the following expression:

Ca×K=Cin×(Ca/Cin)×K (Eq. 8)

From the above Equation 8, Ca/Cin can be thought of as a correction factor for obtaining the true clearance value K0.

Equation 5 above can also be varied such that the following relationship obtains:

Ca/Cin={1−(Cout/Cin)×AR}/(1−AR) (Eq. 9 )

That is, a correction factor for obtaining the true clearance value K0 can be obtained from the above Equation 9, and therefore the true clearance value K0 can be obtained by multiplying this correction factor times the dialyzer 2 particular clearance value K. Thus the clearance value, if used as an indicator during blood purification treatment, can be accurately obtained in real time, thereby enabling the performance of an ideal blood purification treatment which accounts for blood recirculation.

In the present embodiment, as described above, the first solute concentration measuring sensor 15a and the second solute concentration measuring sensor 15b functioning as concentration measurement means are respectively disposed on the arterial blood circuit 1a and the venous blood circuit 1b, but alternatively they may also be disposed on the venous blood circuit 1b only. In such cases, assuming a volume of water Quf removed by the ultrafiltration pump 8 (a known parameter), the true solute concentration Ca can be obtained as noted in what follows.

The following expression is obtained from the definitional equation for the clearance value K:

K={Cin×Qb−Cout×(Qb−Quf)}/Cin (Eq. 10)

From the above Eq. 10, Eq. 6 becomes as noted below, and the true solute concentration Ca can be obtained as:

Ca=[{(Qb−Quf)−(Qb−K)×AR}/{(1−AR)×(Qb−Quf)}]×Cin (Eq. 11)

Above we have explained the present embodiment, but the present invention is not limited thereto, and may, for example, be one in which the recirculation rate derivation means takes a different form (for example, one in which a particular peak is imparted by injecting saline or the like into the venous blood circuit and detecting this peak in the arterial blood circuit to derive a recirculation rate).

Also, in the present embodiment the true blood indicator to be obtained was assumed to be the hematocrit value or solute concentration, but a different true blood indicator (for example, hemoglobin concentration or protein concentration, etc.) can also be used. Other parameters can also be used in lieu of the circulating blood volume rate of change (ΔBV) or clearance value in the embodiments above as parameters derived from this true blood indicator.

For example, the PWI (plasma water indicator), which is an indicator of the degree of influence of water removal-induced body weight change (decrease) in the patient on blood concentrations, can be derived from true blood indicators. This PWI is calculated by a computation which divides the circulating plasma volume change rate (A CPV %) by the patient body weight change rate (ΔBW %): (PWI=ΔCPV %/ΔBW %), and therefore when the estimated dry weight approximates the actual dry weight, verification results make it clear that that numerical value is within the appropriate range.

A high PWI indicates that the rate of blood concentration is high with respect to body weight lost due to water removal, and can be understood to mean that interstitial fluid from outside the blood vessels is not being replenished in comparison to the depletion of water from the blood by water removal, whereas a low PWI can be understood to mean that even if water is depleted from the blood, there is sufficient margin for replenishment of the interstitial fluid.

Moreover, the indicator Kt/V may also be used as another indicator for showing the dialysis efficiency to be derived based on the true blood indicator (solute concentration). That indicator is obtained by the equation below. K indicates clearance value, t is time, and V is distribution volume.

Kt/V=1n(C(0)/C(t)) (Eq. 12)

By substituting the pre-dialysis solute concentration for C(0) and the true solute concentration obtained by the true value derivation means for C(t) in Eq. 12 above, a Kt/V which accounts for blood recirculation can be obtained.

Furthermore, in the present embodiment an ultrafiltration pump is used as a blood concentrating means for imparting a particular peak to the change in blood concentration through the sudden and short duration removal of water, but other means capable of concentrating blood can be used in lieu of the ultrafiltration pump. Moreover, it is also possible to sound a warning when the recirculation blood proportion exceeds a predetermined numerical value, thereby calling the attention of a medical practitioner. In the present embodiment the dialysis device main unit 6 contains a dialysis monitoring device without a built-in dialysate supply mechanism, but the invention may also be applied to a personal use dialysis device with a built-in dialysate supply mechanism.

A blood purification device equipped with a true value derivation means for obtaining a true patient blood indicator based on a recirculation rate obtained by a recirculation rate derivation means may also be applied to other treatments which circulate blood outside the body and perform blood purification (such as blood filtering treatment, blood filtering dialysis treatment, plasma exchange treatment, etc.), or to those with other added functions.

What is claimed is:

1. A blood purification device comprising:
a blood circuit comprising an arterial blood circuit and a venous blood circuit for circulating collected patient blood outside the body;
a blood pump disposed on the arterial blood circuit;
a blood purification means connected between the arterial blood circuit and the venous blood circuit for purifying blood flowing in said blood circuit;
a concentration measurement means for measuring a blood indicator showing the concentration of blood circulating outside the body in the blood circuit;
a recirculation rate derivation means for obtaining a recirculation rate showing the proportion of recirculated blood, which is the volume of blood returned to a patient from the venous blood circuit and once again directed to the arterial blood circuit divided by the volume of blood flowing in the arterial blood circuit; and
a true value derivation means capable of obtaining a patient's true blood indicator based on a recirculation rate obtained by the recirculation rate derivation means.

2. The blood purification device set forth in claim 1, wherein the concentration measurement means is disposed on the blood circuit, and comprises a hematocrit sensor for measuring the hematocrit value of blood flowing in said blood circuit, and the true blood indicator to be obtained by the true value derivation means is a hematocrit value.

3. The blood purification device set forth in claim 2, wherein a circulating blood volume rate of change is calculated as an indicator of patient condition based on the true hematocrit value obtained in the true value derivation means.

4. The blood purification device set forth in claim 1, wherein the concentration measurement means are respectively disposed on the arterial blood circuit and the venous blood circuit of the blood circuit.

5. The blood purification device set forth in claim 1, wherein the blood purification means comprises a dialyzer which either introduces or expels dialysate via a dialysis membrane, and the concentration measurement means can measure a blood indicator showing blood concentration from the dialysate pressure, which is the pressure of dialysate expelled from said dialyzer.

6. The blood purification device set forth in claim 1, wherein the concentration measurement means is disposed on the blood circuit, and comprises a hematocrit sensor for measuring the hematocrit value of blood flowing in said blood circuit and a solute concentration measurement sensor for measuring the solute concentration of blood flowing in said blood circuit, and the true blood indicator to be obtained by the true value derivation means is the solute concentration.

7. The blood purification device set forth in claim 6, wherein the blood purification means comprises a dialyzer which either introduces or expels dialysate via a dialysis membrane, and a clearance value, which is an indicator of the dialysis volume and efficiency of said dialyzer, is calculated based on the true solute concentration obtained by the true value derivation means.

8. The blood purification device set forth in claim 2, wherein the concentration measurement means are respectively disposed on the arterial blood circuit and the venous blood circuit of the blood circuit.

9. The blood purification device set forth in claim 3, wherein the concentration measurement means are respectively disposed on the arterial blood circuit and the venous blood circuit of the blood circuit.
